(19) United States
(12) Patent Application Publication
(10) Pub. No.: US 2011/0208054 A1
(43) Pub. Date: Aug. 25, 2011

(54) ABLATION DEVICE AND METHOD FOR CREATING AN ELONGATE LESION USING SELECTIVELY ACTUATED TRANSDUCER CONTROLLED BY LESION COMPLETION SENSOR

(75) Inventors: Mark T. Stewart, Lino Lakes, MN (US); David E. Francischelli, Anoka, MN (US)

(73) Assignee: Medtronic, Inc.

(21) Appl. No.: 12/712,613

(22) Filed: Feb. 25, 2010

(51) Int. Cl. A61B 18/14; A61B 8/00

(52) U.S. Cl. 600/439; 606/34

(57) ABSTRACT

Device, system and method for evaluating the effectiveness of tissue ablations of a heart of a patient. The tissue is clamped between a pair of opposing jaws. A portion of the tissue is ablated at a first generally linear position on the tissue by applying ablative energy to two of a plurality of elongate electrodes, each of the two of the plurality of elongate electrodes being coupled in opposing relationship to each other and the pair of opposing jaws, respectively. An effectiveness of the ablation is sensed at a second generally linear position on the tissue with at least one of the plurality of elongate electrodes positioned on one of the pair of opposing jaws. The second linear position on the tissue is laterally distal to the first linear position on the tissue with respect to the atrium of the heart.

### FIELD

This disclosure relates to an ablation device and method for creating a lesion and, more particularly, and to such an ablation device and method for creating an elongate lesion.

### BACKGROUND

The action of the heart depends on electrical, and cardiac muscle cell contractile conduction within the heart tissue. In certain people at certain times, electrical signals within heart tissue may not function properly and can create cardiac arrhythmias. Ablation of cardiac conduction pathways in the region of tissue where the signals are malfunctioning may reduce or eliminate such faulty signals. Ablation involves creating lesions on tissue during surgery. To provide effective therapy, surgically created lesions may block the transmission of cardiac contractions.

Ablation may be accomplished in several ways. Sometimes ablation is necessary only at discrete positions along the tissue, is the case, for example, when ablating accessory pathways, such as in Wolff-Parkinson-White syndrome or AV nodal reentrant tachycardias. At other times, however, ablation is desired along a line (either straight or curved), called linear ablation. (In contrast to linear ablation, ablations at discrete positions along the tissue are called non-linear or focal ablations.) One way is to position a tip portion of the ablation device so that an ablation electrode is located at one end of the target lesion line. Then energy is applied to the electrode to ablate the tissue adjacent to the electrode. The tip portion of the electrode is then dragged or slid along the tissue while delivering energy to a new position at the other and of the target lesion line. A second way of accomplishing linear ablation is to use an ablation device having a series of spaced-apart band or coil electrodes that, after the electrode portion of the ablation device has been properly positioned, are energized simultaneously or one at a time to create the desired lesion. If the discrete electrodes are positioned adequately close together, a continuous lesion may be formed.

In addition, electrical pathways through tissue are often not merely or primarily in the surface of the tissue, but rather may run throughout the depth of the tissue. As such, in order to block or cut an adequate number of electrical pathways in order to reduce or prevent electrical propagation, the lesion may need to reach a particular depth in the tissue. In the past, the need to create relatively deep lesions has been addressed by applying ablation energy for relatively long periods of time. In addition, some ablation elements have been developed which allow for a focal zone for ablation energy to be adjusted. When the focal point may be adjusted, relatively deep lesions may be formed by adjusting the focal point to be relatively deeper in the tissue.

Common areas of the heart that are treated using surgically created continuous linear lesions are located in the atria. This may be the case for atrial fibrillation, which is a common form of arrhythmia. The aim of linear ablation in the treatment of atrial fibrillation may be to reduce the total mass of electrically connected atrial tissue below a threshold believed to be needed for sustaining multiple reentry wavelets. Linear transmural lesions may be created between electrically non-conductive anatomic landmarks to reduce the contiguous atrial mass. Transmurality is achieved when the full thickness of the target tissue is ablated.

Before the procedure is complete, the area of the heart may be tested to confirm a conduction block or see if the ablation is effective and eliminates the undesired electrical signals. Present methods to confirm conduction block include the use of electrophysiology catheters to evaluate pulmonary vein isolation lesions and monopolar and bipolar focal probes using pacing or electrogram techniques, and are described below.

### SUMMARY

An ablation system has been developed which may improve the ability to create a transmural lesion in tissue while reducing the time required and avoiding damage to tissue which is undesirable to ablate. In particular, the ablation system may utilize ultrasonic monitoring to monitor the elasticity and/or hydration of the tissue. The less the elasticity and hydration of the tissue, the less time may be required to achieve a transmural lesion. This may reduce the time spent ablating the tissue, as well as reduce the likelihood of excessive ablation being delivered. In addition, the impedance, inductance and/or capacitance of the tissue may be monitored in the target tissue. Based on changes in these electrical characteristics, the effectiveness of the lesion may be determined. In addition, a sensing lead may monitor an amplitude or waveform of an electrogram in the tissue. Based on changes in the amplitude or waveform, further determinations may be made as to the effectiveness of the ablation process so far, as well as further deliveries of ablation energy which may be required.

A controller may factor in the electrical characteristics to make a determination of the effectiveness or completeness of a lesion at a particular location. Based on the determination, the controller may automatically adjust the focal zone of the ablation element to a new depth and continue forming the lesion. The refocusing of the ablation element may continue automatically until the controller determines that the lesion is adequately transmural. In this way, user input may be reduced, as well as time.

Moreover, the controller may be sensitive to tissue which may be undesirable to ablate because the tissue does not naturally propagate electrical energy and because the tissue may be utilized for other, potentially important purposes. Such tissue which may be undesirable to ablate may include blood vessels, nerves and the like. Based on the sensed electrical and physical parameters, the controller may automatically control the focal zone of the ablation element to prevent ablation of tissue which is undesirable to ablate. Such sensed parameters may include Doppler flow detection of coronary arteries or the coronary sinus flow, for example. The controller may direct, focus, focal heating to occur around any such regions of high linear blood flow.

In an embodiment, the present invention provides an ablation device for creating an elongate lesion along a path in tissue of a patient having a controller and a source of ablation energy. An actuatable transducer operatively coupled to the controller and the source of ablation energy, the actuable transducer being movable with respect to the tissue of the patient. A sensor operatively coupled to the controller, the sensor producing an output indicative of at least partial completion of at least a portion of the elongate lesion. The controller controls delivery of ablation energy to a particular portion of the tissue along the path by controlling a position of the actuable transducer along the path based at least partially upon the output of the sensor indicative of a degree of the at least partial completion of at least a portion of the lesion along the path.

In an embodiment, the controller controls the position of the actuable transducer along the path by moving the actuable transducer with respect to the path based at least partially upon a degree of completion of at least a portion of the lesion along the indicated by the output of the sensor.

In an embodiment, a positioning mechanism, the controller controls the position of the actuable transducer on the path by moving the actuable transducer with the positioning mechanism based at least partially upon a degree of completion of at least a portion of the lesion along the indicated by the output of the sensor.

In an embodiment, the positioning mechanism comprises a track positioned with respect to the path and wherein the controller moves the actuable transducer along the track based at least partially upon a degree of completion of at least a portion of the lesion along the path indicated by the output of the sensor.

In an embodiment, the positioning mechanism moves the actuable transducer to one of a plurality of selectable locations on the track based at least partially upon the completion of the lesion indicated by the output of the sensor.

In an embodiment, the controller additionally controls delivery of ablation energy by controlling an amount of the ablation energy delivered by the actuable transducer at a particular location along the path.

In an embodiment, the actuatable transducer has a focal point and wherein the controller controls a distance of the focal point based at least partially upon the completion of the lesion indicated by the output of the sensor.

In an embodiment, the actuable transducer comprises an array of selectively actuable transducer elements.

In an embodiment, the controller controls delivery of ablation energy to a particular portion of the tissue along the path by selectively activating the selectively actuable transducer elements based at least partially upon the completion of the lesion by the output of the sensor.

In an embodiment, the sensor is an ultrasound sensor.

In an embodiment, the condition of the tissue indicated by the ultrasound sensor is indicated by an ultrasound image.

In an embodiment, the sensor is a sensor which senses an acoustical impedance of the tissue by transmitting a sound wave into the tissue and measuring a resistance of the tissue to the sound wave.

In an embodiment, the acoustical impedance of the tissue indicates a hydration of the tissue.

In an embodiment, the acoustical impedance of the tissue indicates an elasticity of the tissue.

In an embodiment, the present invention provides a method for creating an elongate lesion along a path of tissue of a patient using an ablation device having an actuatable transducer operatively coupled to a controller and a source of ablation energy, and a sensor operatively coupled to the controller. The actuable transducer is positioned with respect to the path of the tissue. Ablation energy is delivered to a portion of the path of the tissue with the actuatable transducer. A degree of completion of the lesion in the tissue proximate the portion of the path of the tissue is sensed. The position of the actuable transducer along the path is moved when the degree of completion indicates the lesion proximate the portion of the path of the tissue is complete. Then repeat by returning to deliver ablation energy step until the elongate lesion is complete along an entirety of the path.

In an embodiment, the moving step is controlled by the controller.

In an embodiment, the moving step comprises the controller controls a positioning mechanism coupled to the actuatable transducer.

In an embodiment, the moving step further comprises the positioning mechanism moving the actuable transducer to one of a plurality of selectable locations on a track based on the degree of completion of the lesion indicated by the sensor.

In an embodiment, the delivering ablation energy step further comprises the controller controlling an amount of the ablation energy delivered by the actuable transducer at a particular location along the path.

In an embodiment, the actuatable transducer has a focal point and wherein the delivering ablation energy step further comprises the controller adjusting a distance of the focal point based on the degree of completion of the lesion indicated by the sensor.

In an embodiment, the controller is operatively coupled to the positioning mechanism and where the moving step further comprises the controller controlling the positioning mechanism to position the transducer array based on the degree of completion of the lesion indicated by the sensor.

In an embodiment, the sensor is an ultrasound sensor.

In an embodiment, the sensing step senses a degree of completion of the lesion in the tissue proximate the portion of the path of the tissue based on an ultrasound image generated by the ultrasound sensor.

In an embodiment, the sensor senses an acoustical impedance of the tissue by transmitting a sound wave into the tissue and measuring a resistance of the tissue to the sound wave.

In an embodiment, the acoustical impedance indicates a hydration of the tissue, and wherein the sensing step senses a degree of completion of the lesion in the tissue proximate the portion of the path of the tissue based on the hydration of the tissue.

In an embodiment, the acoustical impedance indicates an elasticity of the tissue, and wherein the sensing step senses a degree of completion of the lesion in the tissue proximate the portion of the path of the tissue based on the elasticity of the tissue.

In an embodiment, the present invention provides a method for creating an elongate lesion along a path of tissue of a patient using an ablation device comprising an actuatable transducer operatively coupled to a controller and a source of ablation energy, and a sensor operatively coupled to the controller. The actuable transducer is selectively actuated at a first selected location along the path. First, a degree of completion of the lesion in the tissue proximate the first selected location along the path of the tissue is sensed. The actuable transducer at the first selected location along the path is deactivated based at least partially upon the degree of completion of the lesion proximate the first selected location obtained in the sensing step. The actuable transducer is selectively actuated at a new selected location along the path based at least partially upon the degree of completion. Then a degree of completion of the lesion in the tissue proximate the new selected location along the path of the tissue is sensed. The actuable transducer is selectively deactivated at the new selected location along the path based at least partially upon the degree of completion of the lesion proximate the first selected location. Then returning to the selectively actuating the actuable transducer at the new selected location step until the elongate lesion is complete along an entirety of the path.

### BRIEF DESCRIPTION OF THE DRAWINGS

FIG. 1 is a schematic drawing of a heart with surgically created lesions;

FIG. 2 is an ablation device for creating a lesion;

FIG. 3 is a side-view of an ablation member of the ablation device of FIG. 2;

FIGS. 4A-4D are front-views of different embodiments of the ablation member of FIG. 3;

FIG. 5 is a block diagram a controller of the ablation device of FIG. 2;

FIGS. 6A and 6B are side-views of an ablation device; and

FIG. 7 is a flowchart of a method of using an ablation device.

### DESCRIPTION

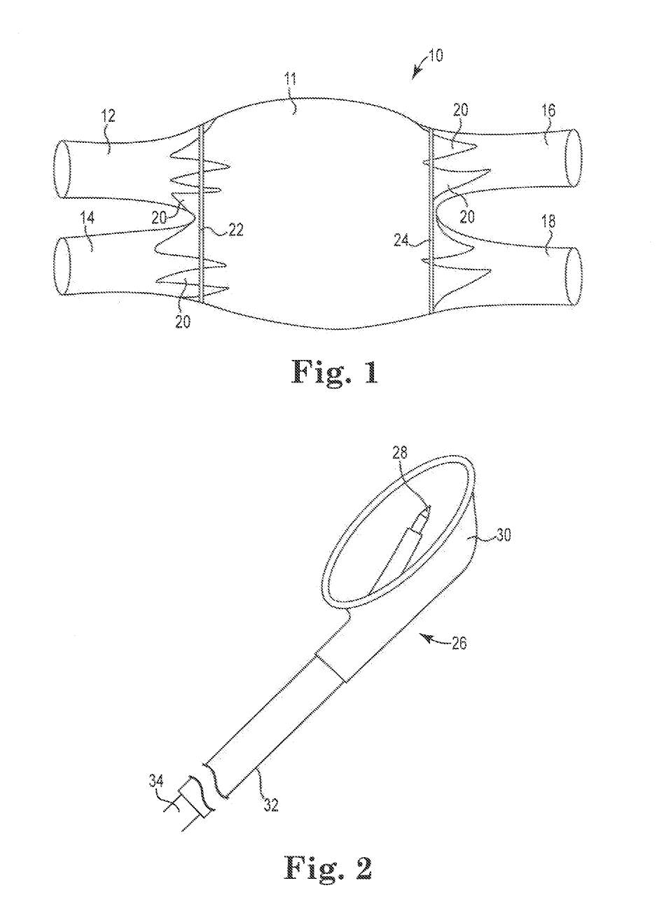

FIG. 1 illustrates a portion of heart 10 as viewed from facing the back of a patient. Heart 10 includes tissue 11 forming left superior pulmonary vein 12, left inferior pulmonary vein 14, right superior pulmonary vein 16 and right inferior pulmonary vein 18. Newly oxygenated blood returns from the lungs into the left atrium through right and left pulmonary veins 12, 14, 16, 18. Heart 10 further includes left atrial myocardium and myocardial extensions 20 onto pulmonary veins 12, 14, 16, 18. In order to treat atrial fibrillation, transmural lesion 22 may be formed on the left atrium (LA), proximal to the left pulmonary veins 12, 14 and transmural lesion 24 may be formed on the left atrium (LA), proximal to the right pulmonary veins 16, 18.

FIG. 2 is an illustration of an embodiment of ablation device 26 (not including a microprocessor 66 nor function generator/amplifier 64) incorporating ablation member 28 positioned on head 30 of neck 32. In an embodiment, neck 32 is flexible, and both head 30 and neck 32 are sized to permit insertion of head 30 and neck 32 through an incision and into the thoracic cavity to a point proximate heart 10. Source of ablation energy 34 is operatively coupled to ablation member 28 by hard-wired connection down neck 32. In alternative embodiments, ablation device 26 does not incorporate neck 32, with ablation member 28 operatively coupled to source of ablation energy 34 by way of other modes known in the art.

In various embodiments, source of ablation energy 34 is a source of ultrasound energy, and ablation member 28 is configured to deliver ultrasound energy. In an embodiment, source of ultrasound energy 34 is a source of high intensity focused ultrasound, known in the art as “HIFU”, and ablation member 28 is configured to deliver high intensity focused ultrasound energy.

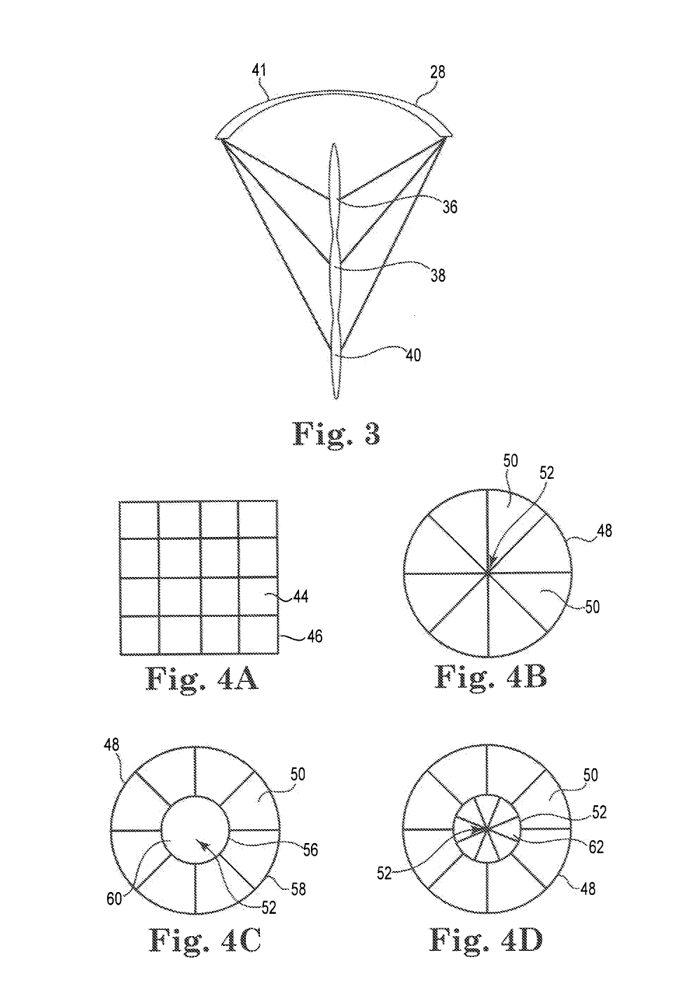

FIG. 3 is a side-view of an embodiment of ablation member 28. In this embodiment, ablation member 28 is a HIFU transducer configured to focus ultrasound energy at adjustable focus zones 36, 38, 40. Focus zones 36, 38, 40 increase in distance from surface 41 of ablation member 28. In various embodiments, ablation member 28 is configured to focus ultrasound energy to discrete focal zones. In such embodiments, the discrete focal zones may have two or more focal zones. In various alternative embodiments, ablation member 28 does not have discrete focal zones, instead allowing a user to adjust the focal point to a variable desired distance from the ablation member.

In the illustrated embodiment, ablation member 28 is an ultrasonic parabolic transducer. The parabolic configuration permits relatively easier focusing of ultrasound energy. In alternative embodiments, ablation member 28 may incorporate alternative profiles as appropriate, including planar, conic and “half-pipe” configurations, half-pipe being related to planar but with two opposing edges curved. In the illustrated embodiment, focal points may be determined on the basis of their distance from surface 41.

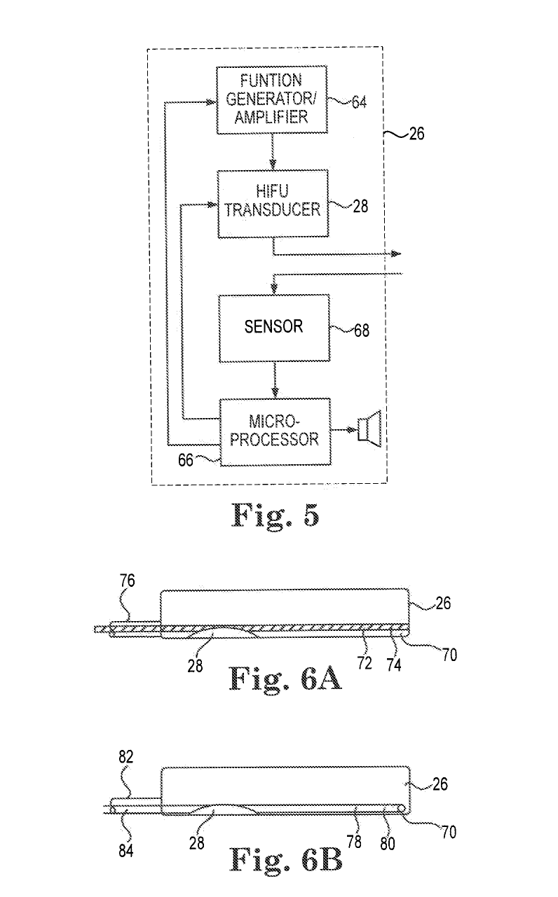

FIGS. 4A-4D are front views of various embodiments of ablation member 28 incorporating multiple independently steerable transducer elements. The perspective drawings of FIGS. 4A-4D are directly perpendicular to surface 41. As such, FIGS. 4A-4D may be utilized in parabolic, planar, “half-pipe” and conic transducers, or any other appropriate transducer.

FIG. 4A is an array of square elements 44. As depicted, square elements 44 are formed into a larger square 46, but may, in alternative embodiments, be formed into any desirable shape comprised of multiple squares. Alternatively, square elements 44 may be rectangles of desirable size.

FIGS. 4B-4D are circular arrays 48 of wedge elements 50. In the embodiment of FIG. 4B, wedge elements 50 extend to center point 52. In the embodiment of FIG. 4C, wedge elements 50 extend only to mid-point 56. In an embodiment, mid-point is two-thirds of the way from edge 58 and center point 52. In alternative embodiments, mid-point is between one-third of the distance from edge 58 to center point 52 and three-quarters of the distance from edge 58 to center point 52. In further alternative embodiments, mid-point 56 is anywhere between edge 58 and center point 52. In an alternative embodiment of FIG. 4C, circular element 60 is positioned in the middle of circular array 48. In the embodiment of FIG. 4D, related to FIG. 4C, wedge elements 50 extend only part-way to center point 52, while center wedge elements 62 occupy the remainder of circular array 48, in general the same area occupied by circular element 60 in FIG. 4C.

All of the embodiments of FIG. 4A-4D may be configured so that transducer elements may be focused at various distances from surface 41 or primary focal point 38. While the various embodiments of FIGS. 4A-4D may be utilized in many different shapes of transducers, as detailed above, certain embodiments of FIGS. 4A-4D may be particularly advantageous in certain circumstances. For instance, while square elements 44 of FIG. 4A may be advantageous in a planar or half-pipe transducer, wedge elements 54 combined with center wedge elements 62 of FIG. 4D may be advantageous in a parabolic transducer.

FIG. 5 is a block diagram of ablation device 26. Ablation element 28 is coupled to function generator/amplifier 64, which supplies ablation energy to ablation element 28. In an embodiment, function generator/amplifier 64 is a source of ablation energy and supplies high intensity focused ultrasound energy to transducer 28. Microprocessor 66 (controller) is coupled to both function generator/amplifier 64 and transducer 28. In an alternative embodiment, microprocessor is coupled only to transducer 28. Microprocessor 66 is operable to control both the delivery of ablation energy from function generator/amplifier 64 and the configuration of ablation element 28, in particular the focal zone. Sensor 68 is coupled to microprocessor 66. Microprocessor may control function generator/amplifier 64 and ablation element 28 on the basis of internal programming and on the basis of feedback from sensor 68.

In various embodiments, pulse-echo sensor 68 senses various acoustic characteristics of the tissue 11 of heart 10 which is being ablated by ablation device 26. In an embodiment, sensor 68 senses elasticity and hydration of the tissue. In alternative embodiments, sensor 68 senses impedance, inductance and/or capacitance of the tissue. In such an embodiment, sensor 68 may incorporate conventional features of commercial frequency analyzers and multi-meters. In further alternative embodiments, sensor 68 senses an electrogram generated by the heart of the patient. In such an embodiment, sensor 68 may incorporate conventional electrogram detection electrodes and hardware well known in the art.

In various embodiments, sensor 68 may incorporate various ones of the above-described detection elements. In such an embodiment, all of the sensor information may be provided to microprocessor 66, which may utilize various combinations of the information in order to control ablation element 28 and function generator/amplifier 64. In an embodiment, sensor 68 may combine a hydration detector, an impedance detector and an electrogram detector, and microprocessor 66 may control the delivery of ablation energy on the basis of the information provided by those detectors.

Data provided to microprocessor 66 by sensor 68 may give microprocessor information regarding the nature of the tissue of heart 10 which is to be ablated. On the basis of that information, ablation energy may be delivered at various intensities and for various lengths of time. For instance, it is possible that it may be desirable in tissue with relatively high hydration and/or relatively high elasticity to ablate at relatively high power for relatively short periods of time. In tissue with relatively low hydration and/or elasticity it may be desirable to ablate at relatively low power for relatively long periods of time.

In addition, microprocessor 66 may determine a thickness of the tissue of heart 10 to be ablated on the basis of data from sensor 68. For instance, atrial tissue which is relatively thick may be greater than five millimeters (5 mm) in thickness may characterize thick tissue. By contrast, tissue which is relatively thin may be less than one millimeter (1 mm) in thickness. Information provided by sensor 68 may be utilized to determine a relatively precise estimate of the thickness of the tissue. On the basis of this determination, microprocessor 66 may thus select an appropriate number of focal zones for ablation element 28 to ablate in order to attain transmurality in the tissue.

In addition, sensor 68 may provide microprocessor 66 information relating to the process towards ablating tissue during an ablation procedure. In particular, when sensor 68 measures impedance and electrogram data, microprocessor 66 may determine progress in forming the lesion. As the lesion becomes relatively more complete, impedance in the tissue tends to rise while the amplitude of the sensed electrograms tends to decrease. As such, in various embodiments, microprocessor 66 may determine that a lesion is complete in a particular location when the measured impedance rises above a certain threshold and the measured electrogram amplitude falls below a certain threshold. In various alternative embodiments, other sensed factors may be utilized in determining that a lesion is complete at a particular location.

Moreover, on the basis of sensed characteristics of the tissue, microprocessor 66 may determine that particular tissue should not be ablated at all. Ablation device 26 may be utilized in locations other than heart 10. Particularly in such circumstances, the tissue to be ablated may include, for instance, blood vessels and nerves, which may be undesirable to ablate due to the physiological impact on the patient. In addition, blood vessels and nerves may be unable to propagate the kinds of electrical signals which are desired to be blocked in ablation. Because tissue such as blood vessels and nerves may possess different characteristics than tissue to be ablated, sensor 68 may provide data to microprocessor 66 which may be utilized by microprocessor 66 to determine that ablation energy should not be applied in certain locations.

In various embodiments, microprocessor 66 may determine that blood vessels or nerves are at particular depths within the target tissue. In such circumstances, microprocessor 66 may control the focal zones at which ablation element 28 delivers ablation energy to ablate tissue, for instance, above and below a blood vessel or nerve, but not ablate the blood vessel or nerve itself.

As illustrated in FIG. 1, it may be desirable to create an elongate lesion 22, 24 in tissue 11 of heart 10. In circumstances where lesion 22, 24 need not be elongate, ablation device 26 may be positioned once and microprocessor 66 may control the delivery of ablation energy and the focal zone of ablation element 28 in order to create a discrete transmural lesion. In circumstances where an elongate lesion may be desirable, various embodiments of ablation device 26 may provide it without a user having to manually move ablation device 26. In an embodiment incorporating an array of square elements 44, square elements 44 may be configured in an elongate configuration sized to create the desired lesion. In an embodiment, square elements 44 may be formed in the “half-pipe” configuration to enhance the creation of a focal zone.

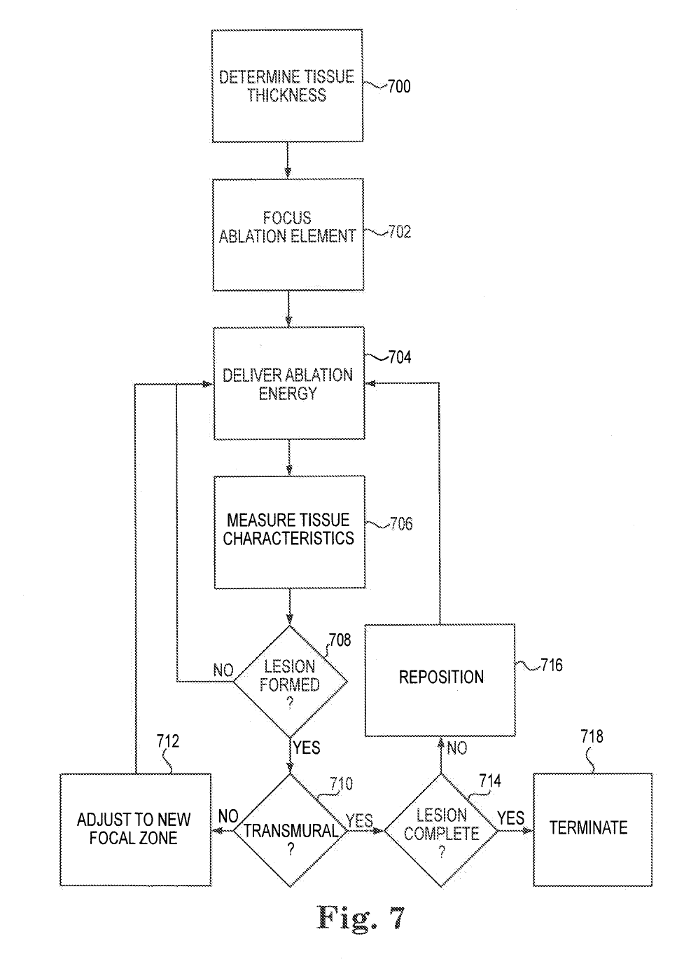

FIGS. 6A and 6B show an alternative embodiment of ablation device 26 which incorporates ablation element 28 attached to automatic repositioning system 70. In such embodiments, ablation element 28 may be an ablation element 28 of many different sizes and configurations, and may be moved to different linear positions in order to create a linear transmural lesion.

In FIG. 6A ablation element 28 is coupled to screw drive 72. Screw drive 72 functions in a manner common to screw drives known in the art. By actuating screw 74 clockwise and counterclockwise, ablation element 28 moves up and down screw 74. Screw 74 is coupled to motor 76 which provides motive power to turn screw 74. In an embodiment, motor 76 is coupled to microprocessor 66, which may initiate movement of screw 74 and thus ablation element 28 on the basis of data from sensor 68.

In FIG. 6B ablation element 28 is connected to cable drive 78. Cable drive 78 functions in a manner common to cable drives known in the art. Cable 80 is wound around pulley 82 and is coupled to motor 84. When motor 84 moves cable 80 ablation element 28 moves with respect to pulley 82. As with motor 76, motor 84 is, in an embodiment, coupled to and controlled by microprocessor 66.

Alternative embodiments of devices which may move ablation element 28 to different locations are contemplated. In cases involving automatic repositioning, ablation element 28 may be positioned at a first location, at which a transmural lesion may be created by varying the focal zone until transmurality is achieved. Once transmurality is achieved in the first location, ablation element 28 is repositioned to a second location, where a second transmural location is created. Additional transmural locations may be created at additional locations, such that ultimately, once all transmural lesions have been created, the transmural lesions are in contact with each other in order to create a single elongate transmural lesion. In alternative embodiments, ablation element 28 may be steadily moved among various locations, repeating visits to various locations as a transmural lesion is gradually formed over the length of the elongate lesion. In automatic repositioning embodiments, a user of ablation device 26 may program microprocessor 66 with a desired length of the elongate transmural lesion.

In various alternative embodiments, ablation device may be configured to curvilinear lesions. In an embodiment, cable drive 78 may be adapted to curve, with cable 80 pulling ablation element 28 in a curved pattern. In such an embodiment, the curved elongate lesion may be formed in the same manner as described with respect to the linear elongate lesion described above. In various embodiments, ablation device 26 may be reconfigurable by attaching a new automatic repositioning system 70. Alternatively, automatic reposition system 70 may be capable of having its shape changed. In such an embodiment, automatic repositioning system 70 may be flexed or otherwise adjusted into various shapes.

In various further embodiments, repositioning system 70 is not automatic but manually controlled by a user. In such an embodiment, ablation device 26 may provide a prompt to a use to manipulate repositioning system 70 to reposition ablation element 28. The user prompt may be any conventional prompt known in the art, including but not limited to a tone or other sound, a light or other visual indicator, or a vibration or other mechanical output.

FIG. 7 is a flowchart of a method for ablating tissue utilizing ablation device 26. Tissue thickness is determined (700) at a tissue location. Ablation element 28 is focused (702) by microcontroller 66 to a focal zone and ablation energy is delivered (704) from function generator/amplifier 64. Sensor 68 measures (706) characteristics of tissue 11, and microcontroller 66 determines (708) if an appropriate lesion has been formed at the current focal zone. If not, ablation energy is delivered (704). If the lesion has been fanned at the focal zone, microcontroller 66 determines (710) if the lesion is transmural by referencing data from sensor 68. If the lesion is not transmural the focal zone is adjusted (712) to a new focal zone and ablation energy is delivered (704). If the lesion is transmural then microcontroller 66 determines (714) if the lesion is complete. If the lesion is not complete then ablation element 28 is repositioned (716) to a new location and ablation energy is delivered (704). If the lesion is complete then the ablation procedure terminates (718).

In various alternative embodiments, the above procedure may be varied dependent on the circumstances. For instance, it may be desirable to first reposition (716) ablation element 28 rather than adjusting (712) the focal zone. In such an embodiment, repositioning (716) may be swapped with adjusting (712), and the flowchart followed normally. In further alternative embodiments, ablation element 28 could be mounted on a robotically controlled manipulator for minimally invasive access.

Although the present invention has been described with reference to preferred embodiments, workers skilled in the art will recognize that changes can be made in form and detail without departing from the spirit and scope of the present invention.

What is claimed is:

1. An ablation device for creating an elongate lesion along a path in tissue of a patient, comprising:
a controller;
a source of ablation energy;
an actuatable transducer operatively coupled to said controller and said source of ablation energy, said actuable transducer being movable with respect to said tissue of said patient;
a sensor operatively coupled to said controller, said sensor producing an output indicative of at least partial completion of at least a portion of said elongate lesion;
wherein said controller controls delivery of ablation energy to a particular portion of said tissue along said path by controlling a position of said actuable transducer along said path based at least partially upon said output of said sensor indicative of a degree of said at least partial completion of at least a portion of said lesion along said path.

2. The ablation device of claim 1 wherein said controller controls said position of said actuable transducer along said path by moving said actuable transducer with respect to said path based at least partially upon a degree of completion of at least a portion of said lesion along said indicated by said output of said sensor.

3. The ablation device of claim 2 further comprising a positioning mechanism, said controller controlling said position of said actuable transducer on said path by moving said actuable transducer with said positioning mechanism based at least partially upon a degree of completion of at least a portion of said lesion along said indicated by said output of said sensor.

4. The ablation device of claim 3 wherein said positioning mechanism comprises a track positioned with respect to said path and wherein said controller moves said actuable transducer along said track based at least partially upon a degree of completion of at least a portion of said lesion along said path indicated by said output of said sensor.

5. The ablation device of claim 4 further wherein said positioning mechanism moves said actuable transducer to one of a plurality of selectable locations on said track based at least partially upon said completion of said lesion indicated by said output of said sensor.

6. The ablation device of claim 1 wherein said controller additionally controls delivery of ablation energy by controlling an amount of said ablation energy delivered by said actuable transducer at a particular location along said path.

7. The ablation device as in claim 6 wherein said actuatable transducer has a focal point and wherein said controller controls a distance of said focal point based at least partially upon said completion of said lesion indicated by said output of said sensor.

8. The ablation device of claim 1 wherein said actuable transducer comprises an array of selectively actuable transducer elements.

9. The ablation device of claim 8 wherein said controller controls delivery of ablation energy to a particular portion of said tissue along said path by selectively activating said selectively actuable transducer elements based at least partially upon said completion of said lesion by said output of said sensor.

10. The ablation device of claim 1 wherein said sensor is an ultrasound sensor.

11. The ablation device of claim 10 wherein said condition of said tissue indicated by said ultrasound sensor is indicated by an ultrasound image.

12. The ablation device of claim 1 wherein said sensor is a sensor which senses an acoustical impedance of said tissue by transmitting a sound wave into said tissue and measuring a resistance of said tissue to said sound wave.

13. The ablation device of claim 12 wherein said acoustical impedance of said tissue indicates a hydration of said tissue.

14. The ablation device of claim 12 wherein said acoustical impedance of said tissue indicates an elasticity of said tissue.

15. A method for creating an elongate lesion along a path of tissue of a patient using an ablation device comprising an actuatable transducer operatively coupled to a controller and a source of ablation energy, and a sensor operatively coupled to said controller, comprising the steps of:
positioning said actuatable transducer with respect to said path of said tissue; then
delivering ablation energy to a portion of said path of said tissue with said actuatable transducer;
sensing a degree of completion of said lesion in said tissue proximate said portion of said path of said tissue with said sensor; then
moving a position of said actuatable transducer along said path when said degree of completion indicates said lesion proximate said portion of said path of said tissue is complete; and
returning to said delivering ablation energy step until said elongate lesion is complete along an entirety of said path.

16. The method of claim 15 wherein said moving step is controlled by said controller.

17. The method of claim 16 wherein said moving step comprises said controller controlling a positioning mechanism coupled to said actuatable transducer.

18. The method of claim 17 wherein said moving step further comprises said positioning mechanism moving said actuable transducer to one of a plurality of selectable locations on a track based on said degree of completion of said lesion indicated by said sensor.

19. The method of claim 15 wherein said delivering ablation energy step further comprises said controller controlling an amount of said ablation energy delivered by said actuable transducer at a particular location along said path.

20. The method of claim 19 wherein said actuatable transducer has a focal point and wherein said delivering ablation energy step further comprises said controller adjusting a distance of said focal point based on said degree of completion of said lesion indicated by said sensor.

21. The method of claim 17 wherein said controller is operatively coupled to said positioning mechanism and where said moving step further comprises said controller controlling said positioning mechanism to position said transducer array based on said degree of completion of said lesion indicated by said sensor.

22. The method of claim 15 wherein said sensor is an ultrasound sensor.

23. The method of claim 22 wherein said sensing step senses a degree of completion of said lesion in said tissue proximate said portion of said path of said tissue based on an ultrasound image generated by said ultrasound sensor.

24. The method of claim 15 wherein said sensor senses an acoustical impedance of said tissue by transmitting a sound wave into said tissue and measuring a resistance of said tissue to said sound wave.

25. The method of claim 24 wherein said acoustical impedance indicates a hydration of said tissue, and wherein said sensing step senses a degree of completion of said lesion in said tissue proximate said portion of said path of said tissue based on said hydration of said tissue.

26. The method of claim 24 wherein said acoustical impedance indicates an elasticity of said tissue, and wherein said sensing step senses a degree of completion of said lesion in said tissue proximate said portion of said path of said tissue based on said elasticity of said tissue.

27. A method for creating an elongate lesion along a path of tissue of a patient using an ablation device comprising an actuatable transducer operatively coupled to a controller and a source of ablation energy, and a sensor operatively coupled to said controller, comprising the steps of:
selectively actuating said actuable transducer at successive selected locations along said path; then
sensing a degree of completion of said lesion in said tissue proximate each one of said successive selected locations along said path of said tissue; then
deactivating said actuable transducer at said one of said successive selected locations along said path based at least partially upon said degree of completion of said lesion proximate said one of said successive selected locations; then
returning to said selectively actuating said actuable transducer at said successive selected locations along said path step until said elongate lesion is complete along an entirety of said path.
